SEQUENCE LISTING (23 sequences)

SEQ ID NO: 1 - DNA, 52 bp, homo sapiens
  misc_feature (27)..(27): n = a or g
cattctcatcagtattttaattccagntatgttgtttccttgataattttct

SEQ ID NO: 2 - DNA, 52 bp, homo sapiens
  misc_feature (27)..(27): n = c or t
ttccccatattccccacccagcactcnacctctttagctcttcaggtctcag

SEQ ID NO: 3 - DNA, 51 bp, homo sapiens
  misc_feature (26)..(26): N = a or g
tagggtcccgccggtcaagatggtcnccccggacccccaggcccacctggt

SEQ ID NO: 4 - DNA, 52 bp, homo sapiens
  misc_feature (27)..(27): n = c or g
aycctcagggccccagcctggagtggntgaagaaactgtgagcacctccact

SEQ ID NO: 5 - DNA, 52 bp, homo sapiens
  misc_feature (27)..(27): n = c or t
tggattccatatcccactgccaaaacngcatggttcagattatcgctattgc

SEQ ID NO: 6 - DNA, 52 bp, homo sapiens
  misc_feature (27)..(27): n = c or g
gtggctactggtgctgacgcctggccngccggccgcgggactatccacctgc

SEQ ID NO: 7 - DNA, 52 bp, homo sapiens
  misc_feature (27)..(27): n = c or t
acatcatgggacagagaaatccgatgnggtttgcagttcttctctgccagct

SEQ ID NO: 8 - DNA, 52 bp, homo sapiens
  misc_feature (27)..(27): n = c or t
gatccggatcaggatgcaacatacttnggggcttttaaagttcgagatatag

SEQ ID NO: 9 - DNA, 52 bp, homo sapiens
  misc_feature (27)..(27): n = c or t
ctgctgccttccctgccccagtacccncaggagaagattccaaagatgtagc

SEQ ID NO: 10 - DNA, 52 bp, homo sapiens
  misc_feature (27)..(27): n = c or t
tggcctgcttgctgttcttacaggganggaggcaatggcggccagcacttcc

SEQ ID NO: 11 - DNA, 52 bp, homo sapiens
  misc_feature (27)..(27): n = c or t
cctggggtgcaggacgccgcgctgatngaggccatccaggaccgcctgtcca

SEQ ID NO: 12 - DNA, 52 bp, homo sapiens
  misc_feature (27)..(27): n = a, c, g or t
gttccccaactgttttgaattctagtngcagaatggatgaatctgtttggcg

SEQ ID NO: 13 - DNA, 52 bp, homo sapiens
  misc_feature (27)..(27): n = c or t
ggaataatcaatgaagcattcaattcngtcatcttgctccactctcccattt

SEQ ID NO: 14 - DNA, 52 bp, homo sapiens
  misc_feature (27)..(27): n = a or g
ctctggggctggtagaattgctatttntccatgatcaagagcaccactctta

SEQ ID NO: 15 - DNA, 52 bp, homo sapiens
  misc_feature (27)..(27): n = g or t
ccgccaccccacctgcccagggaatgngggcgggatgagggctggacctccc

SEQ ID NO: 16 - protein, 113 aa, homo sapiens
MIPAKDMAKVMIVMLAICFLTKSDGKSVKKRSVSEIQLMHNLGKHLNSMERVEWLRKKLQDVHNFVALGAPLAPRDAGSQRPRKKEDNVLVESHEKSLGEADKADVNVLTKAK

SEQ ID NO: 17 - protein, 110 aa, homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 18 - protein, 233 aa, homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 19 - protein, 167 aa, homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 20 - protein, 100 aa, homo sapiens
MRALTLLALLALAALCIAGQAGAKPSGAESSKGAAFVSKQEGSEVVKRPRRYLYQWLGAPVPYPDPLEPRREVCELNPDCDELADHIGFQEAYRRFYGPV

SEQ ID NO: 21 - protein, 372 aa, homo sapiens
ETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQV

SEQ ID NO: 22 - protein, 273 aa, homo sapiens
MRIAVICFCLLGITCAIPVKQADSGSSEEKQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 23 - protein, 212 aa, homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

(19) United States
(12) Patent Application Publication
(10) Pub. No.: US 2011/0166107 A1
(43) Pub. Date: Jul. 7, 2011

(54) METHODS AND KITS FOR DETECTING RISK FACTORS FOR DEVELOPMENT OF JAW OSTEONECROSIS AND METHODS OF TREATMENT THEREOF

(75) Inventors: Joseph Katz, Gainesville, FL (US); Taimour Y. Langaee, Gainesville, FL (US)

(73) Assignee: UNIVERSITY OF FLORIDA RESEARCH FOUNDATION INC., Gainesville, FL (US)

(21) Appl. No.: 12/937,953

(22) PCT Filed: Jul. 7, 2009
(86) PCT No.: PCT/US09/49767

§ 371 Date: Mar. 14, 2011

Related U.S. Application Data
(60) Provisional application No. 61/078,680, filed on Jul. 7, 2008.

(51) Int. Cl. A61K 31/663; C12Q 1/68; C40B 30/04; C40B 40/06; A61P 19/08

(52) U.S. Cl. 514/108; 435/6.11; 506/9; 506/16

(57) ABSTRACT

Methods of and kits for determining the pharmacogenetic, pharmacokinetic and cellular basis of bisphosphonate-induced osteonecrosis of the jaw (BONJ) involve associating particular proteins and particular single nucleotide polymorphisms with a risk for developing BONJ after receiving bisphosphonate treatment. Methods and kits for identifying the genetic basis for a patient's predisposition to BONJ, and methods of identifying patients who are prone to develop BONJ following bisphosphonate administration provide for the development of a tool for physicians to prescribe treatment protocols for BONJ patients based on the patients' genomes (“personal/tailored medicine”).

### CROSS REFERENCE TO RELATED APPLICATION

The present application claims the priority of U.S. provisional patent application No. 61/078,680 filed Jul. 7, 2008.

### FIELD OF THE INVENTION

The invention relates generally to the fields of molecular biology, genetics, and medicine. More particularly, the invention relates to genetic polymorphisms and protein expression in serum useful for assessing jaw osteonecrosis risks in humans receiving or prescribed bisphosphonates.

### BACKGROUND OF THE INVENTION

Bisphosphonates are prescribed to alleviate bone pain, bone destruction and hypercalcemia in many cancer patients, or to reduce bone loss in osteoporotic individuals. The bisphosphonates are a class of drugs that inhibit the activities and functions of osteoclasts (bone resorbing cells) and perturb the differentiation of osteoblasts (bone forming cells). There are two types of bisphosphonates: nitrogen-containing and non nitrogen-containing. Bisphosphonates inhibit bone resorption and thus bone renewal by suppressing the recruitment and activity of osteoclasts thus shortening their life span. IV bisphosphonates are primarily used to treat bone erosion and hypercalcemia associated with bone metastasis from multiple myeloma and other malignancies. Oral bisphosphonates are also used to prevent bone loss and are prescribed for patients with osteoporosis. Painful exposure of bone in the jaws of patients receiving the bisphosphonates pamidronate (Aredia®; Novartis Pharmaceuticals) and zoledronate (Zometa®; Novartis Pharmaceuticals) was first reported by Marx in 2003. Since then, several authors have reported additional cases and many dental professionals, particularly oral and maxillofacial surgeons have identified numerous unpublished cases.

Bisphosphonates are commonly prescribed to stabilize bone loss caused by osteoporosis in millions of postmenopausal women. The strategy in the treatment of osteoporosis is to inhibit the resorption of trabecular bone by osteoclasts and hence preserve its density. For this purpose, oral bisphosphonates are prescribed and include etidronate (Didronel®; Procter and Gamble), risedronate (Actonel®; Procter and Gamble), tiludronate (Skelid®; Sanofi-Synthe Lab Inc), and alendronate (Fosamax®; Merck). More potent bisphosphonates are delivered intravenously and are indicated to stabilize metastatic cancer (primarily breast and prostate) deposits in bone, and to treat the bone resorption defects of multiple myeloma and correct severe hypercalcemia. These are pamidronate and zoledronate. In addition to the drugs mentioned here, several other bisphosphonates are known that are either not commonly used in the United States or that remain experimental.

Recent reports suggest that there is an association between the use of bisphosphonates and osteonecrosis of the jaw. Bisphosphonate-induced osteonecrosis of the jaw (BONJ) is a morbid bone disorder in a subset of patients. The physiological mechanisms by which this complication manifests in bisphosphonate users are still unknown. Because the jaws have a greater blood supply than other bones and a faster bone turnover rate related both to their daily activity and the presence of teeth (which mandates daily bone remodeling around the periodontal ligament), bisphosphonates are highly concentrated in the jaws. Coupled with chronic invasive dental diseases and treatments and the thin mucosa over bone, this anatomic concentration of bisphosphonates causes this condition to be manifested exclusively in the jaws. Thus, the exposed bone in the jaws is the direct result of the action of these bisphosphonates on the daily remodeling and replenishment of bone. Osteoblasts and osteocytes live for only about 150 days. If, upon their death, the mineral matrix is not resorbed by osteoclasts, which release the cytokines of bone morphogenetic protein and insulin-like growth factors to induce new osteoblasts from the stem cell population, the osteon becomes acellular and necrotic. The small capillaries within the bone become involuted, and the bone becomes a-vascular. A spontaneous breakdown of the overlying mucosa, some form of injury, or an invasive surgery to the jaws usually causes this necrotic bone to become exposed which then fails to heal.

The majority of BONJ cases seen and reported were patients treated with IV bisphosphonates. While up to 13% of patients receiving IV bisphosphonates develop BONJ, estimates for oral bisphosphonates are 1:10,000 to 1:100,000. Although a controlled, randomized, prospective, blinded study to prove the specific causal relationship between bisphosphonate therapy and exposed bone is not possible, the drugs pamidronate, zoledronate, and more rarely alendronate have shown a direct correlation that cannot be ignored.

Most BONJ cases to date are diagnosed in cancer patients with bone metastases. However, BONJ cases have been also reported after oral therapy for osteoporosis. Thus, a large proportion of the general population (i.e. post menopausal women and cancer patients) may be at risk.

### SUMMARY OF THE INVENTION

Described herein are methods of identifying the genetic basis for a patient's predisposition to BONJ, methods and kits for identifying patients who are prone to develop BONJ following bisphosphonate administration, and to the development of tools for physicians to prescribe treatment protocols for BONJ patients based on the patients' genomes (“personal/tailored medicine”) and serum protein expression. Also described herein are proteins whose expression in serum or saliva can be associated with BONJ and detected by known methods (e.g., Western blots, ELISA, etc.). The methods described herein include identifying genes that contribute to the development of BONJ using a candidate gene approach. A case-control design study is used that involves studying the effects of genes (e.g., expression and particular SNP(s)) and serum protein expression in patients with and without BONJ.

Accordingly, described herein is a method of identifying a subject having a predisposition to BONJ following bisphosphonate administration. The method includes the steps of: (a) obtaining a sample from the subject; (b) analyzing the sample for the presence of at least one gene having an SNP that is a biomarker for BONJ or a predisposition to BONJ, a protein encoded by the at least one gene, or at least one SNP that is a marker for BONJ or a predisposition to BONJ; and (c) correlating the presence of the gene, protein or SNP that is a marker for BONJ or a predisposition to BONJ in the sample with a predisposition to BONJ in the subject. The sample can include, for example, blood, serum, plasma or saliva. The at least one SNP can be rs12458117 (SEQ ID NO:1) or rs243865 (SEQ ID NO:2). The step of analyzing the sample for the presence of at least one gene having an SNP that is a biomarker for BONJ or a predisposition to BONJ, a protein encoded by the at least one gene, or at least one SNP that is a marker for BONJ or a predisposition to BONJ can include use of a microarray to detect the presence of the at least one gene or at least one SNP.

Also described herein is a method of preventing BONJ in a subject having a predisposition to BONJ and receiving bisphosphonate therapy. The method includes the steps of: (a) obtaining a sample from the subject; (b) analyzing the sample for the presence of at least one gene having an SNP that is a biomarker for BONJ or a predisposition to BONJ; a protein encoded by the gene, or at least one SNP that is a biomarker for BONJ or a predisposition to BONJ; (c) correlating the presence of the at least one gene, protein, or at least one SNP that is a biomarker for BONJ or a predisposition to BONJ in the sample with a predisposition to BONJ in the subject; and (d) administering to the subject a bisphosphonate that is not associated with BONJ or that is less likely to cause BONJ than other bisphopsphonates, administering to the subject a bisphosphonate at a lower dose or lower frequency than what is conventionally prescribed. The sample can include, for example, blood, serum, plasma or saliva. The at least one SNP can be rs12458117 (SEQ ID NO:1) or rs243865 (SEQ ID NO:2). The step of analyzing the sample for the presence of at least one gene having an SNP that is a biomarker for BOND or a predisposition to BONJ, a protein encoded by the at least one gene, or at least one SNP that is a marker for BOND or a predisposition to BONJ can include use of a microarray to detect the presence of the at least one gene or at least one SNP.

Further described herein is a kit for identifying patients who have a predisposition to BOND following biphosphonate administration. The kit includes (a) a solid support having a plurality of nucleic acids adhered thereto, wherein at least one of the nucleic acids specifically hybridizes to a gene having an SNP that is a biomarker for BONJ or a predisposition to BONJ; (b) a detection reagent; and (c) instructions for use. The at least one gene can be a gene set forth in Table 1 or Table 3, and the SNP can be an SNP set forth in Table 1 or Table 3.

Also described herein is a method for assessing a subject's risk of developing BOND following bisphosphonate treatment includes a) obtaining a biological sample from the subject; b) detecting one or more BONJ-associated biomarkers in said sample, wherein the biomarkers are related to one or more genes set forth in Table 1 or Table 3, or said biomarkers are related to one or more polypeptides encoded by said genes resulting in a biomarker data set; c) comparing the biomarker data set to biomarker data from healthy people and people having BOND; and d) determining the subject's risk of developing BOND. In the method, at least one biomarker can be an SNP residing in a gene set forth in Table 1 or Table 3, a BONJ-associated polymorphic site associated with one or more of the SNP markers set forth in Table 1 or Table 3, or an expression product of a gene set forth in Table 1 or Table 3. At least one biomarker can be an SNP being in complete linkage disequilibrium with one or more of the SNP markers set forth in Table 1 or Table 3.

Still further described herein is a method of identifying a subject having a predisposition to BOND following bisphosphonate administration. The method includes the steps of: (a) obtaining a sample from the subject; (b) analyzing the sample for expression of a protein involved in bone homeostasis; and (c) correlating expression of the protein with a predisposition to BONJ in the subject. The protein involved in bone homeostasis can be one of: PTH, insulin, TNF-α, leptin, OPN, OC, OPG and IL6. The sample can be analyzed for overexpression of the protein. In the method, the protein can be one that shares at least 90% amino acid sequence identity with a protein listed in Table 4.

Unless otherwise defined, all technical terms used herein have the same meaning as commonly understood by one of ordinary skill in the art to which this invention belongs.

As used herein, a “nucleic acid,” “nucleic acid molecule,” or “polynucleotide” means a chain of two or more nucleotides such as RNA (ribonucleic acid) and DNA (deoxyribonucleic acid). A “purified” nucleic acid molecule is one that has been substantially separated or isolated away from other nucleic acid sequences in a cell or organism in which the nucleic acid naturally occurs (e.g., 30, 40, 50, 60, 70, 80, 90, 95, 96, 97, 98, 99, 100% free of contaminants). The term includes, e.g., a recombinant nucleic acid molecule incorporated into a vector, a plasmid, a virus, or a genome of a prokaryote or eukaryote.

By the term “gene” is meant a nucleic acid molecule that codes for a particular protein, or in certain cases, a functional or structural RNA molecule. For example, the COL 1A 1 gene encodes the COL1A1 protein.

As used herein, “protein” or “polypeptide” are used synonymously to mean any peptide-linked chain of amino acids, regardless of length or post-translational modification, e.g., glycosylation or phosphorylation.

When referring to a nucleic acid molecule or polypeptide, the term “native” refers to a naturally-occurring (e.g., a WT) nucleic acid or polypeptide.

By the terms “osteopontin protein” or “OPN” or “OPN polypeptide” is meant an expression product of an OPN gene such as a protein that shares at least 65% (but preferably 75, 80, 85, 90, 95, 96, 97, 98, or 99%) amino acid sequence identity with native human osteopontin (OPN) protein and displays a functional activity of a native OPN protein. A “functional activity” of a protein is any activity associated with the physiological function of the protein. For the additional proteins described herein (e.g., parathyroid hormone (PTH), insulin, TNF-a, osteocalcin (OC), osteoprotegerin (OPG), etc.), similar meanings of terms relating to these additional proteins are meant.

As used herein, “sequence identity” means the percentage of identical subunits at corresponding positions in two sequences when the two sequences are aligned to maximize subunit matching, i.e., taking into account gaps and insertions. Sequence identity is present when a subunit position in both of the two sequences is occupied by the same nucleotide or amino acid, e.g., if a given position is occupied by an adenine in each of two DNA molecules, then the molecules are identical at that position. For example, if 7 positions in a sequence 10 nucleotides in length are identical to the corresponding positions in a second 10-nucleotide sequence, then the two sequences have 70% sequence identity. Sequence identity is typically measured using sequence analysis software (e.g., Sequence Analysis Software Package of the Genetics Computer Group, University of Wisconsin Biotechnology Center, 1710 University Avenue, Madison, Wis. 53705).

A “biomarker” in the context of the present invention refers to a SNP marker disclosed in Tables 1 or 3 or to a polymorphism of a gene disclosed in Tables 1 or 3 or at a locus closely linked thereto, or to an organic biomolecule which is related to a gene set forth in Tables 1 or 3 and which is differentially present in samples taken from subjects (patients) having BONJ compared to comparable samples taken from subjects who do not have BONJ. An “organic biomolecule” refers to an organic molecule of biological origin, e.g., steroids, amino acids, nucleotides, sugars, polypeptides, polynucleotides, complex carbohydrates or lipids. A biomarker is differentially present between two samples if the amount, structure, function or biological activity of the biomarker in one sample differs in a statistically significant way from the amount, structure, function or biological activity of the biomarker in the other sample.

A “haplotype,” as described herein, refers to any combination of genetic markers (“alleles”). A haplotype can include two or more alleles and the length of a genome region including a haplotype may vary from a few hundred bases up to hundreds of kilobases. As it is recognized by those skilled in the art, the same haplotype can be described differently by determining the haplotype defining alleles from different nucleic acid strands. The haplotypes described herein are differentially present in individuals with BONJ or having an increased risk of BONJ than in individuals without BONJ. Therefore, these haplotypes have diagnostic value for risk assessment, diagnosis and prognosis of BONJ or risk of BONJ in an individual. Detection of haplotypes can be accomplished by methods known in the art used for detecting nucleotides at polymorphic sites. The haplotypes described herein, e.g. having markers such as those shown in Tables 1 or 3 are found more frequently in individuals with BONJ or having an increased risk of BONJ than in individuals without BONJ. Therefore, these haplotypes have predictive value for detecting BONJ or a susceptibility (increased risk) to BONJ in an individual.

A nucleotide position in a genome at which more than one sequence is possible in a population, is referred to herein as a “polymorphic site” or “polymorphism”. Where a polymorphic site is a single nucleotide in length, the site is referred to as a SNP. For example, if at a particular chromosomal location, one member of a population has an adenine and another member of the population has a thymine at the same position, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a thymine allele.

The SNP markers having novel BONJ associations as described herein (e.g., in Tables 1 or 3) have official reference SNP (rs) ID identification tags assigned to each unique SNP by the National Center for Biotechnological Information (NCBI). Each rs ID has been linked to specific variable alleles present in a specific nucleotide position in the human genome, and the nucleotide position has been specified with the nucleotide sequences flanking each SNP. For example the COL1A1 SNP having rs ID rs180012 is in chromosome 17, variable alleles are A and C, and the nucleotide sequence assigned to rs180012 is SEQ ID NO: 3:

As used herein, an “allele” may refer to a nucleotide at a SNP position (wherein at least two alternative nucleotides are present in the population at the SNP position, in accordance with the inherent definition of a SNP) or, for cSNPs, may refer to an amino acid residue that is encoded by the codon which contains the SNP position (where the alternative nucleotides that are present in the population at the SNP position form alternative codons that encode different amino acid residues). An “allele” may also be referred to herein as a “variant”. Also, an amino acid residue that is encoded by a codon containing a particular SNP may simply be referred to as being encoded by the SNP.

“Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize to its specific target. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “non-specific priming sequences” or “nonspecific sequences” to which the primer or probe has varying degrees of complementarity. Probes and primers may include modified bases as in polypeptide nucleic acids. Probes or primers typically include about 15 to 30 consecutive nucleotides and they may further include a detectable label, e.g., radioisotope, fluorescent compound, enzyme, or enzyme co-factor. Probes and primers to a SNP marker disclosed in Tables 1 and 3 are either commercially available or easily designed using the flanking nucleotide sequences assigned to a SNP rs ID and standard probe and primer design tools. Primers and probes for SNP markers disclosed in Tables 1 and 3 can be used in risk assessment as well as in molecular diagnostic methods and kits as described herein.

The terms “arrays,” “microarrays,” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. Microarrays can be prepared and used by a number of methods, including those described in U.S. Pat. No. 5,837,832 (Chee et al.), PCT application WO95/11995 (Chee et al.), Lockhart, D. J. et al. (Nat. Biotech. 14:1675-1680, 1996) and Schena, M. et al. (Proc. Natl. Acad. Sci. 93:10614-10619, 1996), all of which are incorporated herein in their entirety by reference. In other embodiments, such arrays can be produced by the methods described by Brown et al., U.S. Pat. No. 5,807,522.

By the phrases “risk genes,” “risk genes for diseases,” and “disease loci” is meant genetic variants that confer an increased likelihood of developing disease.

The terms “patient,” “subject” and “individual” are used interchangeably herein, and mean a mammalian (e.g., human) subject to be treated and/or to obtain a biological sample from.

Although methods and kits similar or equivalent to those described herein can be used in the practice or testing of the present invention, suitable methods and kits are described below. All publications, patent applications, patents and other references mentioned herein are incorporated by reference in their entirety. In the case of conflict, the present specification, including definitions will control. The particular embodiments discussed below are illustrative only and not intended to be limiting.

### BRIEF DESCRIPTION OF THE DRAWINGS

FIG. 1 is a photograph of the mouth of a man suffering from painful, non-healing necrotic bone exposure in the mandible of a multiple myeloma patient treated with Zometa® (zoledronate, Novartis).

### DETAILED DESCRIPTION OF THE INVENTION

The invention relates to identifying risk factors for developing BONJ in a patient or subject (e.g., humans) receiving bisphosphonate treatment or potentially receiving bisphosphonate treatment. Described herein are methods of determining the pharmacogenetic, pharmacokinetic and cellular basis of BONJ. Also described herein are methods and kits for identifying the genetic basis for a patient's predisposition to BONJ, and methods of identifying patients who are prone to develop BONJ following bisphosphonate administration provide for the development of tools for physicians to prescribe treatment protocols for BONJ patients based on the patients' genomes (“personal/tailored medicine”). A haplotype tagging SNP approach was used to analyze candidate genes involved in bone absorption and destruction and to examine the influence of genetic variants on the susceptibility of BONJ. Bone biomarkers of BONJ were examined using molecular cell techniques. The methods described herein can be used to identify differences in how patients are genetically predisposed to BONJ as well as genetic differences amongst patients that account for differences in how these patients clear bisphosphonates from their systems. Determining such genetic differences provides for improved monitoring of the drugs used to treat BONJ, improved prevention of BONJ, and optimized treatment of such individuals.

The below described preferred embodiments illustrate adaptations of these methods and kits. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Methods for performing SNP and haplotype analyses as well as genotyping techniques are described, for example, in Computational Methods for SNPs and Haplotypes, 1^{st} ed., S. Istrail et al., Springer Press, Totowa, N.J., 2004; N. Maniatis Methods Mol. Biol. 376:109-121, 2007; Genetic Analysis of Complex Disease by Jonathan L. Haines and Margaret A. Pericak-Vance, 2^{nd} ed., 2006, Wiley-Liss Publishing, Hoboken, N.J.; and Single Nucleotide Polymorphisms Methods and Protocols (Methods in Molecular Biology) by Pui-Yan Kwok, 1^{st} ed., 2002, Humana Press, New York, N.Y. Genome-wide association studies are reviewed in Pearson and Manolio, JAMA vol. 299:1335-1344, 2008.

### Single Nucleotide Polymorphisms (SNPs)

The coexistence of multiple forms of a genetic sequence gives rise to genetic polymorphisms, including SNPs. SNPs are single base positions in DNA at which different alleles, or alternative nucleotides, exist in a population, and are the most common form of genetic variation in the genome. The SNP position (interchangeably referred to herein as SNP, SNP site, SNP locus, SNP marker, biomarker, or marker) is usually preceded by and followed by highly conserved sequences of the allele (e.g., sequences that vary in less than 1/100 or 1/1000 members of the populations). An individual may be homozygous or heterozygous for an allele at each SNP position. In some embodiments, an SNP is referred to as a “cSNP” to denote that the nucleotide sequence containing the SNP is an amino acid coding sequence.

A SNP may arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP may also be a single base insertion or deletion variant referred to as an “indel” (Weber et al., Am. J. Hum. Genet. 71:854-62, 2002).

As used herein, references to SNPs and SNP genotypes include individual SNPs and/or haplotypes, which are groups of SNPs that are generally inherited together. Haplotypes can have stronger correlations with diseases or other phenotypic effects compared with individual SNPs, and therefore may provide increased diagnostic accuracy in some cases. Causative SNPs are those SNPs that produce alterations in gene expression or in the expression, structure, and/or function of a gene product, and therefore are most predictive of a possible clinical phenotype. One such class includes SNPs falling within regions of genes encoding a polypeptide product, i.e. cSNPs. These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Such variant products can result in a pathological condition, e.g. genetic disease.

Causative SNPs do not necessarily occur in coding regions; causative SNPs can occur in, for example, any genetic region that can ultimately affect the expression, structure, and/or activity of the protein encoded by a nucleic acid. Such genetic regions include, for example, those involved in transcription, such as SNPs in transcription factor binding domains, SNPs in promoter regions, in areas involved in transcript processing, such as SNPs at intron-exon boundaries that may cause defective splicing, or SNPs in mRNA processing signal sequences such as polyadenylation signal regions. Some SNPs that are not causative SNPs nevertheless are in close association with, and therefore segregate with, a disease-causing sequence. In this situation, the presence of a SNP correlates with the presence of, or predisposition to, or an increased risk in developing the disease. These SNPs, although not causative, are nonetheless also useful for diagnostics, disease predisposition screening, and other uses.

Although the numerical chromosomal position of a SNP may still change upon annotating the current human genome, the SNP identification information such as variable alleles and flanking nucleotide sequences assigned to a SNP will remain the same. Those skilled in the art will readily recognize that the analysis of the nucleotides present in one or more SNPs set forth in Tables 1 and 3 of this invention in an individual's nucleic acid can be done by any method or technique capable of determining nucleotides present in a polymorphic site using the published sequence information to the rs IDs of the SNPs listed in Tables 1 and 3. The nucleotides present in polymorphisms can be determined from either nucleic acid strand or from both strands.

It is understood that the BONJ-associated SNP markers and haplotypes described in Tables 1 and 3 may be associated with other polymorphisms present in the same BONJ-associated genes and loci as described herein. TagSNPs are loci that can serve as proxies for many other SNPs. The use of tagSNPs greatly improves the power of association studies as only a subset of loci needs to be genotyped while maintaining the same information and power as if one had genotyped a larger number of SNPs. These other polymorphic sites associated with the SNP markers listed in Tables 1 and 3 of this invention may be either equally useful as biomarkers or even more useful as causative variations explaining the observed BONJ-association of SNP markers and haplotypes as described herein.

Also described herein are isolated peptides and polypeptides encoded by genes listed in Tables 1 and 3 including polymorphic positions (e.g., SNPs) disclosed herein. In one embodiment, the peptides and polypeptides are useful screening targets to identify drugs for treating BONJ.

### Identifying Genes Involved in Osteonecrosis of the Jaw

Described herein are methods of identifying genes involved in BONJ. To identify such genes, the haplotype tagging SNPs from the International Hapmap project are examined in order to construct meaningful haplotypes. SNPs are also selected for study based on published reports of positive associations with drug response or disease, known or potential functional effects (i.e. nonsynonymous cSNPs), and allele frequencies. Since SNPs with identical allele frequencies are often in complete linkage disequilibrium, SNPs with varying allele frequencies are selected so as to capture a greater portion of the gene's variability.

In a typical method of identifying genes and SNPs that are involved in BONJ, genes and SNPs that are thought to play a role in osteoclastogenesis, osteoclast differentiation, and bone resorption, bone mineral density (BMD); osteoclast-mediated bone resorptionin tissues of patients with BONJ; and aggressive periodontitis are analyzed. For example, the genes listed in Table 1 are genes and SNPs that are thought to play a role in osteoclastogenesis, osteoclast differentiation, and bone resorption, bone mineral density (BMD); osteoclast-mediated bone resorptionin tissues of patients with BONJ; and aggressive periodontitis, and were reported to be important and significant. These genes are described below.

Osteoclast Associated. Receptor (OSCAR) which is expressed specifically on osteoclast-lineage cells regulates osteoclastogenesis (Kim et al., J Exp Med 195:201-209, 2002). OSCAR may be an important bone-specific regulator of osteoclast differentiation. Multiple alternatively spliced transcript variants encoding different isoforms have been found for this gene.

Variants have been identified in the OSCAR gene (Hallman et al., Metabolism 53:1184-1191, 2004). The SNP A>G at −2322 of the 5′ flanking (promoter) region of OSCAR gene showed significant association with bone mineral density (BMD) in postmenopausal women (Kim et al., J Bone Miner Res. 20:1342-1348, 2005).

Cathepsin K is a lysosomal cysteine protease involved in bone remodeling and resorption. This protein, which is a member of the peptidase Cl protein family, is predominantly expressed in osteoclasts. However, the encoded protein is also expressed in a significant fraction of human breast cancers, where it could contribute to tumor invasiveness. Mutations in this gene are the cause of pycnodysostosis, an autosomal recessive disease characterized by osteosclerosis and short stature (Saftig et al., Proc Natl Acad Sci USA 95:13453-13458, 1998; Haagerup et al., Eur J Hum Genet 8:431-436, 2000).

In a recent study by Hansen T, et al. (Hansen et al., Virchows Arch. 449(4):448-454, 2006), the role of cysteine proteinase cathapsin K in osteoclast-mediated bone resorptionin tissues of patients with BONJ was investigated. This study verified increased numbers of osteoclasts in patients suffering from BONJ. Although it is known that bisphosphonates decrease osteoclast function, these findings suggest a critical involvement of osteoclasts in the mechanisms of bone destruction in the respective lesions. Hansen et al. also showed that the numbers and activity of osteoclast significantly increases in infected osteoradionecrosis and BONJ compared to control tissues. It was concluded that osteoclasts are involved in the process responsible for bone destruction in jaw necrosis.

TGFB is a multifunctional peptide that controls proliferation, differentiation, and other functions in many cell types. TGF β1 which is encoded by TGFB1 gene is the most abundant growth factor in human bone. TGF β1 is produced by osteoblasts and inhibits osteoclast proliferation and activity. It also functions as a regulator of susceptibility to osteoporosis and has been shown to affect on both osteoblast and osteoclast function in vitro (Massague J and Chen Y G., Genes & Dev. 14:627-644, 2000; Langdahl et al., Bone 32:297-310, 2003).

Human macrophage-specific colony-stimulating factor (CSF-1) along with Receptor Activator of NF-KB Ligand (RANKL) are involved in activation and differentiation of monocyte subsets into osteoclasts (Rabello et al., Biochem Biophys Res Commun. 347:791-796, 2006; Komano et al., Arthritis Res Ther. 8:R152, 2006). A recent study of Japanese population showed a positive association between aggressive periodontitis and three polymorphisms located in CSF1.

Osteoclastogenesis in vivo is regulated by action of osteoblast/stromal cells that express membrane-bound, receptor activator of NF-kB ligand (RANKL). RANKL, a member of TNF family, is a cytokine which is essential for induction of osteoclastogenesis. Both osteoblasts and stromal cells produce this cytokine and the signal is transduced by specific receptor called RANK that is localized on the surface of osteoclast progenotors (Boyle et al., Nature 423:337-342, 2003). Both RANKL and CSF-1 are essential for stimulation and differentiating the monocytes into osteoclasts (Komano et al., Arthritis Res Ther 8:R152, 2006). Studies suggest that osteoclasts are involved in bone erosion and inhibition of osteoclastogenesis by controlling the RANKL can limit bone destruction in experimental model of arthritis (Walsh et al., Immunol Rev. 208:228-251, 2005). Koh J M et al. 2006 identified that two novel polymorphisms (+34863G>A and +35928 insdelC) in RANK that may be possible genetic factor for low BMD in postmenopausal women (Koh et al., Osteoporos Int 2006). In another study by Hsu Yh, et al. 2006 showed significant positive associations with BMD in men and polymorphisms in exon 7 (Ala192Val) of the RANK and 5′ UTR of the RANKL genes (Hsu et al., Hum Genet 118:568-577, 2006).

The COL1A1 gene is considered as an important functional candidate for the pathogenesis of osteoporosis because the type I collagen is the major protein of bone and mutation in this gene results in syndrome called osteogenesis imperfecta which is characterized by reduced BMD and increase bone fragility (Boyde et al., Calcif Tissue Int. 64:185-190, 1999). Genetic variants may play important role in osteoporosis or osteoporotic fractures by affecting the metabolism of COL1A1 gene. A study by Yamada (Yamada et al., Hum Biol. 77:27-36, 2005) showed that a G>T polymorphism at −1997 in the promoter of the COL1A1 gene to be associated with BMD for the lumbar spine in postmenopausal Spanish women. Another study showed that the G>T polymorphism that affect a binding site for Sp1 transcription factor in the first intron of COL1A1 gene, the T allele was associated with osteoporosis (Grant et al., Nat Genet. 14:203-205, 1996). In a functional analysis study by Mann V, et al., it was shown that T allele of the Sp1 polymorphism is associated with increased transcription and abnormally increased production of the COL1A1 mRNA and protein (Mann et al., J Clin Invest. 107:899-907, 2001). Ralson S H and colleagues, in a large prospective meta-analysis study (GENOMOS) in more than 20,000 participants showed an association between homozygote T allele of the Sp1 polymorphism and BMD and incident vertebral fracture (Ralston et al., PLoS Med. 3:e90, 2006).

IL-6 is a pleiotropic cytokine that plays a critical role in bone resorption. Ferrari et al. showed that a G>C polymorphism at −174 in the promoter region of IL-6 gene has a functional effect in vivo and affects gene transcription and results in decreasing of circulating levels of IL-6 (Ferrari et al., Arthritis Rheum. 44:196-201, 2001). It was also shown that the haplotype of two allelic variants in the IL-6 promoter region −572 and −174 G was associated with bone resorption markers (Ferrari et al., J Clin Endocrinol Metab. 88:255-259, 2003).

The vitamin D receptor (VDR), a steroid receptor, acts as a transcriptional factor which responds to steroid vitamin D hormone. Vitamin D regulates bone cell differentiation, osteoblast differentiation, bone turnover and calcium homeostasis by interaction with vitamin D receptor (Haussler et al., J Bone Miner Res. 13; 325-349, 1998).

The VDR gene was one of the first genes that were studied in relation to osteoporosis. A large and comprehensive study on VDR and its relation to osteoporosis (the Rotterdam study) was conducted on 6418 individuals by Fang (Fang et al., Am J Hum Genet. 77:807-823, 2005). The authors used the haplotype-tagging SNPs approach and analyzed 15 haplotype-tagging SNPs, and showed that haplotype alleles in the promoter and 3′ UTR regions of VDR gene were associated with increased risk of osteoporotic fracture, and subjects who carried both risk alleles had significantly higher risk (48%) of developing fracture when compared with control individuals. The combination of risk haplotypes results in lower VDR mRNA expression level caused by decreased transcription and increased mRNA degradation (Fang et al., Am J Hum Genet. 77:807-823, 2005).

Runt-related transcription factor 2 (RUNX2), also known as CBFA1 gene, encodes a protein that binds to osteoblast-specific cis-acting element and plays an essential role in the regulation of osteoblast differentiation and inducing osteoblast-specific transcripts, like the one encoding osteocalci (Ducy et al., Cell 89:747-754, 1997; Schinke et al., J Biol Chem. 274:30182-30189, 1999). Several studies reported the association of RUNX2 polymorphisms with BMD. A recent study identified 16 allelic variations within the RUNX2 gene and promoters (P1 and P2) (Doecke et al., J Bone Miner Res. 21:265-273, 2006). The polymorphisms are located within the promoter or polyalanine and polyglutamine repeats of exon 1. In this study, it was shown that the polymorphisms in the promoter region affect RUNX2 transcription in a reporter assay, and are associated with BMD. It was also shown that polymorphisms in the RUNX2 gene that affect BMD are in linkage disequilibrium.

After identifying any genes related to BONJ, SNPs are tagged (about 10 for each gene) and then genotyped to create haplotypes. Genes that are shown to be statistically significant are analyzed for single SNPs. Genomic DNA (gDNA) is isolated from whole blood using any suitable extraction method, e.g., the QIAamp DNA Blood kit from Qiagen (Valencia, Calif.). Isolated gDNA is stored at −20° C. (e.g., in bar-coded 1.5 ml microcentrifuge tubes). Isolated gDNA is quantified by any suitable method, such as a spectrophotometric method using a 96 well plate reader. An aliquot of the stock DNA sample for each participant is transferred from 1.5 μl microcentrifuge tubes to bar-coded 96 well microtiter plates and normalized to 20 ng/ul, using a liquid handling robotic system or other suitable system. Stock genomic DNA samples contain bar codes for identification, as do the 96-well plates. All are managed in the freezer using Freezerworks version 5.3 software. This software allows for precise tracking of the location of every sample within the freezer and the volume of the sample that remains.

TABLE 1
Candidate Genes for Bisphosphonate-associated Osteronecrosis of the Jaw

| | HUGO | Chromosomal | |
|---|---|---|---|
| Name | Gene Name | location | NCBI SNP IDs |
| Osteoclast Associated Receptor | OSCAR | 19q13.4 | rs4147630ⁿ (SEQ ID NO: 4) |
| Cathepsin K | CTSK | 1q21 | rs10788796^{s} (SEQ ID NO: 5) |
| Transforming Growth Factor, Betal | TGFB1 | 19q13.1 | rs1800471ⁿ (SEQ ID NO: 6) |
| Receptor Activator of NF-_{κ}B | TNFRSF11 | 18822.1 | rs1805034ⁿ, (SEQ ID NO: 7) |
| | A (RANK) | | |
| Receptor Activator of NF-_{κ}B ligand | TNFSF11 | 13q14 | rs9562415^{s} (SEQ ID NO: 8) |
| | (RANKL) | | |
| Collagen, type I, ALPHA-1 | COL1A1 | 17q21.31-q22 | rs1800211ⁿ, (SEQ ID NO: 3) |
| Interleukin-6 | IL6 | 7p21 | rs2069830ⁿ, (SEQ ID NO: 9) |
| Vitamin D Receptor | VDR | 12q12-q14 | rs2228570ⁿ (SEQ ID NO: 10), |
| | | | rs731236^{s} (SEQ ID NO: 11) |
| Runt-Related Transcription Factor 2 | RUNX2 | 6p21 | rs11498198ⁿ (SEQ ID NO: 12) |

ⁿdenotes SNPs resulting in a nonsynonymous change,
^{s}denotes SNPs resulting in a synonymous change

In a typical method, one of three PCR-based genotyping methods are utilized. Selection of PCR primers and conditions is optimized through use of a suitable primer design software (e.g., Oligo Primer Analysis Software Version 6).

In a typical method, the genotyping platform used for genotyping is Taqman. The ABI Taqman Prism 7900 HT Sequence Detection System is a second generation, real-time quantitative PCR system with high-throughput capability that can use either 96 or 384-well microtiter plates and a Micro Fluidic Card. This system is also equipped with new software for large-scale genotyping of known SNPs that produces reliable and reproducible results at low cost. Applied Biosystems (Foster City, Calif.) has over 180,000 validated Taqman® SNP genotyping assays, with an additional 1.6 million predesigned assays for non-coding SNPs. Custom assays for SNPs not in their assay library can also be developed.

Pyrosequencing is a real-time DNA sequencing technique that involves hybridization of a sequencing primer to single-stranded, PCR-amplified DNA, along with various substrates and enzymes. The sequencing primers are designed by using special primer design software provided by Pyrosequencing. Upon incorporation of nucleotide(s) into a nucleic acid chain by DNA polymerase, an equimolar quantity of inorganic pyrophosphate is released and subsequently converted to ATP by ATP sulfurylase. The ATP drives a luciferase reaction where luciferin molecule is oxidized to produce light. The light is captured on a charge coupled device camera and seen as a peak on a pyrogram. The pyrosequencing reaction generates sequence data of 20-30 bp and allows genotyping with >99% accuracy and reproducibility. The PSQ HS 96A system supports throughput of up to 3,000 genotypes per workday. In another method, genotyping is achieved by restriction fragment length polymorphism (RFLP) analysis.

### Identifying A Patient Having A Predisposition to BONJ

The invention provides a method for identifying a patient or subject (e.g., human) that is predisposed to or at risk of BONJ following bisphosphonate administration. In a typical embodiment, an individual who is at risk for BONJ is an individual in whom one or more BONJ-associated polymorphisms selected from Tables 1 or 3 are identified and/or expression (e.g., overexpression) of one or more of the proteins listed in Table 4 (or precursors, mature forms, or cleavage fragments thereof) is detected in their serum. In other embodiments, polymorphisms associated with SNPs and haplotypes of Tables 1 or 3 and/or serum expression of proteins listed in Table 4 (or precursors, mature forms, or cleavage fragments thereof) may be used in risk assessment of BONJ. Patients who are candidates for biphosphonate treatment are screened for the presence of the specific gene or SNP or protein. Patients who test positive can be considered for an alternative treatment or otherwise complete a complete dental treatment prior to the biphosphonate therapy.

A typical method of identifying a subject having a predisposition to BONJ following bisphosphonate treatment includes obtaining a sample from the patient; analyzing the sample for the presence of at least one gene having an SNP that is a biomarker for BONJ or a predisposition to BONJ, a protein encoded by the gene, or at least one SNP that is a biomarker for BONJ or a predisposition to BONJ; and correlating the presence of the at least one gene, protein, or at least one SNP that is a biomarker for BONJ or a predisposition to BONJ in the sample with a predisposition to BONJ in the subject. Any appropriate sample can be obtained, e.g., blood, serum, plasma, saliva, etc. As described in the Examples below, SNPs rs12458117 (SEQ ID NO:1) and rs243865 (SEQ ID NO:2) were shown to be present at a higher rate in patients having BONJ than in patients without BONJ. Thus, in one example of a method of identifying a patient or subject (e.g., human) that is predisposed to or at risk of BONJ following bisphosphonate administration, the subject's sample is analyzed for the presence of SNPs rs12458117 (SEQ ID NO:1) and rs243865 (SEQ ID NO:2). The subject's sample can be analyzed for the presence of such SNPs using any suitable method, including use of a microarray. In addition, several methods and different genotyping platforms that are used for SNP genotyping are known in the art (e.g., TaqMan, Pyrosequencing, RFLP, Direct Sequencing, etc.). Microarrays or Chips usually contain thousands up to a million or a little over one million SNPs, which are also used for genotyping.

Alternatively or in addition to analyzing a subject's sample for the presence of particular SNPs, a subject's sample can be analyzed for the presence of a gene in which a particular SNP resides, or a protein encoded by a gene in which a particular SNP resides. Methods of analyzing a sample for the presence of a gene or a protein are well known in the art, and are described in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Such methods include PCR for verifying the presence of a gene and Real Time PCR is also used for detecting the presence of a protein in different cells or tissues

### Treating a Patient Having BONJ

Methods of treating a patient having BONJ are described herein. Some biphosphonates are prone to less side effects like BONJ. For instance Aredia® is less likely to cause BONJ compared to Zometa®. Thus, patients who express the specific genes associated with BONJ are treated with drugs that are less likely to cause BONJ. For example, Zometa® would not be used, its frequency of use would be reduced, and/or its dosage would be reduced. In another example, one would use only Aredia®.

### Kits

Described herein are kits for identifying patients who are prone to develop BONJ following biphosphonate administration. In vitro test kits (e.g. reagent kits) for identifying patients who are prone to develop BONJ following biphosphonate administration include reagents, materials and protocols for assessing one or more biomarkers (e.g., nucleic acids, proteins), and instructions and optionally software for comparing the biomarker data from a subject to biomarker data from healthy and diseased people to make risk assessment, a diagnosis or a prognosis of BONJ. Useful reagents and materials for kits include, but are not limited to PCR primers, hybridization probes and primers as described herein (e.g., labeled probes or primers), allele-specific oligonucleotides, reagents for genotyping SNP markers, reagents for detection of labeled molecules, restriction enzymes (e.g., for RFLP analysis), DNA polymerases, RNA polymerases, DNA ligases, marker enzymes, microarrays, antibodies which bind to altered or to non-altered (native) BONJ risk gene encoded polypeptides, means for amplification of nucleic acids fragments from one or more BONJ-risk genes selected from Tables 1 and 3, means for analyzing the nucleic acid sequence of one or more BONJ risk genes or fragments thereof, or means for analyzing the sequence of one or more amino acid residues of a BONJ risk gene-encoded polypeptide, etc.

A typical kit for identifying patients who are prone to developing BONJ following biphosphonate administration includes a solid support having a plurality of nucleic acids adhered thereto (e.g., a nucleic acid array), wherein at least one (e.g., 1, 2, 3, 4, 5, 6, 7, 8, 9, 10) of the nucleic acids specifically hybridizes to a gene having an SNP that causes BONJ or a predisposition to BONJ; a detection reagent; and instructions for use. Generally, the solid support will have two or more (e.g., 2, 3, 4, 5, 6, 7, 8, 9, 10) nucleic acids adhered thereto that specifically hybridize to two or more (e.g., 2, 3, 4, 5, 6, 7, 8, 9, 10) genes having SNPs that are markers for a predisposition to BONJ. In one embodiment, a kit includes a blood test and/or saliva test for the expression of specific genes and SNPs that are associated with BONJ. In another embodiment, a kit for diagnosing or predicting susceptibility to BONJ includes primers and reagents for detecting the nucleotides present in one or more SNP markers selected from Tables 1 and 3 in an individual's nucleic acid. Information obtained from use of kits as described herein can be used to optimize treatment of individuals having BONJ or suspected of having BONJ.

Another example of a kit includes reagents for detecting the presence of one or more proteins (e.g., proteins listed in Table 4, or precursors, mature forms, or cleavage fragments thereof) whose expression in serum can be used to assess an individual's risk of developing BONJ. Such a kit can include the reagents and instructions necessary for carrying out a Western blot, for example, or an ELISA.

### EXAMPLES

### Example 1

### Determination of Allele Frequencies for SNPs

Methods

Genomic DNA was isolated from lymphocytes in whole blood using a commercially available kit (Qiagen DNA Blood Isolation Kit, Qiagen, Valencia, Calif.). The isolated DNA samples were quantified by spectrophotometry and agarose gel electrophoresis methods and standardized to 20 ng/ul.

Genotyping for the two alleles SNPs SNP [A/C], dbSNP ID (rs1800012) (SEQ ID NO:15), COL1A1 gene chromosome 17, and SNP [A/G], dbSNP ID (rs12458117) (SEQ ID NO:1), TNFRSF11A gene chromosome 18 was performed by PCR, and by the fluorescence-based TaqMan® (Applied Biosystems, Foster City, USA) genotyping method (De la Vega et al., Mutat Res. 573(1-2):111-135, 2005).

TaqMan genotyping assay probes [(C_{—}7477174_{—}30 for SNP [A/C], dbSNP ID (rs1800012) (SEQ ID NO:15), COL1A1 gene chromosome 17, and C_{—}31393804 for SNP [A/G], dbSNP ID (rs12458117) (SEQ ID NO:1), TNFRSF11A gene chromosome 18] were purchased from Applied Biosystems, Foster City, USA. The probes are fluorescent dyes (VIC and FAM) labeled primers designed by ABI (Applied Biosystems) for genotyping.

Preliminary Results

After genotyping the six samples for the above mentioned two SNPs [SNP [A/C], dbSNP ID (rs1800012) (SEQ ID NO:15), COL1A1 gene chromosome 17, and SNP [A/G], dbSNP ID (rs12458117) (SEQ ID NO:1), TNFRSF11A gene chromosome 18], the minor allele frequency (MAF) was determined to be 17% for both SNPs.

SNP [A/C], dbSNP ID (rs1800012) (SEQ ID NO:15), COL1A1 gene C allele=17%

SNP [A/G], dbSNP ID (rs12458117) (SEQ ID NO:1), TNFRSF11A gene G allele=17%

The association between BONJ and dbSNP ID (rs 1800012) (SEQ ID NO:15) and dbSNP ID (rs12458117) (SEQ ID NO:1), was significant statistically.

### Example 2

### Genotyping for SNPs

Genomic DNA was isolated from lymphocytes of blood samples from 50 subjects, 6 cases of BONJ and 45 controls (patients without BONJ) and were genotyped for 4 single nucleotide polymorphisms (SNPs): dbSNP ID (rs1934980 (SEQ ID NO:13), and rs1934951 (SEQ ID NO:14)) from the CYP2C8 gene; dbSNP ID (rs1800012) (SEQ ID NO:15) from the COL1A1 gene, and dbSNP ID (rs12458117) (SEQ ID NO:1), from the TNFRSF11A gene.

Total number of cases and controls genotyped = 48
(cases = 6, controls = 42)

| | | |
|---|---|---|
| rs1934980 (SEQ ID NO: 13) | SNP A/G | CYP2C8 gene |
| A = 82% | | |
| G = 18% | | |
| G allele in cases = 2 | | |
| % G allele in controls = 16% | | |
| rs1934951 (SEQ ID NO: 14) | SNP C/T | CYP2C8 gene |
| C = 78% | | |
| T = 22% | | |
| T allele in cases = 2 | | |
| % T allele in controls = 20% | | |
| rs1800012 (SEQ ID NO: 15) | SNP A/C | COL1A1 gene |
| A = 93% | | |
| C = 7% | | |
| C allele in cases = 1 | | |
| % C allele in controls = 6% | | |
| rs12458117 (SEQ ID NO: 1) | SNP G/A | TNFRSF11A gene |
| G = 87% | | |
| A = 13% | | |
| A allele in cases = 2 | | |
| % A allele in controls = 11% | | |

### Example 4

### Analysis of Candidate Gene SNPs Association with BONJ

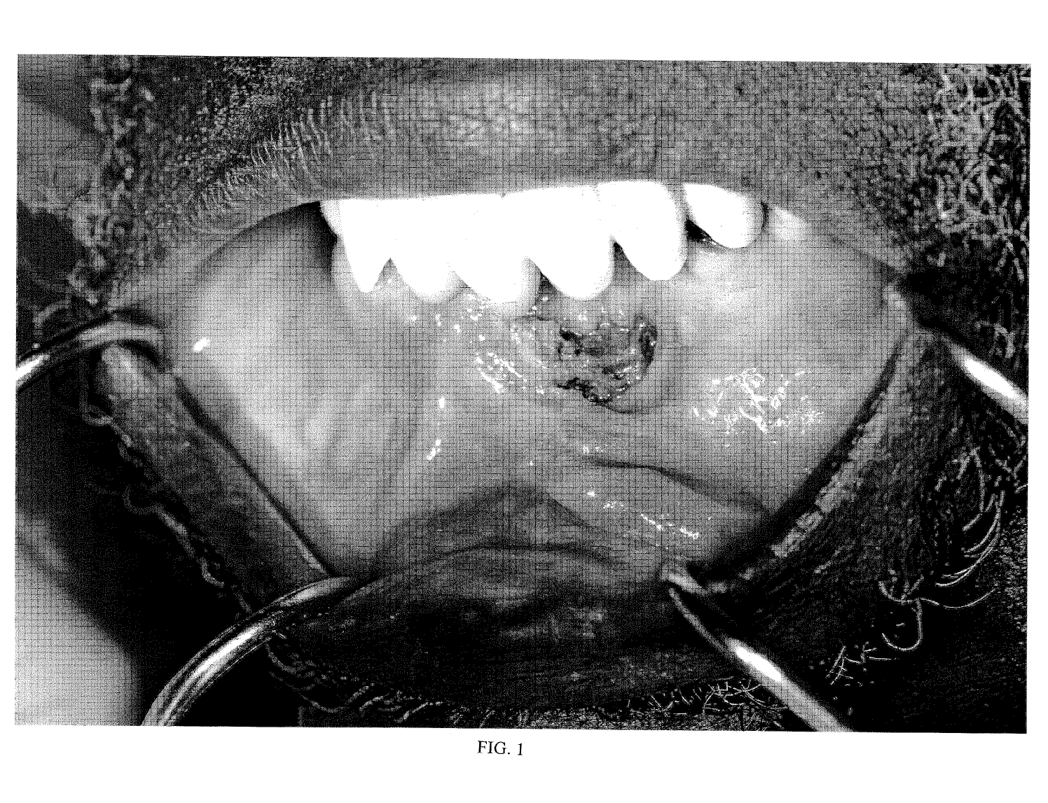

Medical and dental charts at the University of Florida (UF) and the associated Veterans Administration Medical Center (VAMC) were reviewed. As shown in Table 2, 27 patients were identified with BONJ having a median age of 62 years, 19 with myeloma, 3 with prostate cancer, 2 with breast cancer, 2 with head and neck cancers, and one with renal cell carcinoma. There were 21 males. Twelve patients received sequential pamidronate and zoledronate treatments, eleven had zoledronate and 3 had pamidronate. Fourteen patients had modest increases in their serum creatinine concentrations. The average number of previous chemo/radiotherapy regimens was 3.5. Nine patients had Thalidomide, 5 had Bortizomib. Primary disease status was as follows: 12 patients in clinical remission, 5 with stable disease and 10 with progressive disease. Eight patients received statin therapy and 6 had diabetes mellitus. The median length of treatment with BP before the diagnosis of BONJ was 28 months. BONJ involved the mandible in 21 patients, maxilla in 4 and both in 2 patients. The most frequent presentation was pain, swelling, and exposed bone (FIG. 1). Ten patients had a preceding dental procedure. The BONJ incidence differed among the 3 centers where these patients were treated. In the outpatient bone marrow transplant clinic where all patients had myeloma, the total incidence was 13%; while it was 4% for pamidronate only. The incidence in the VAMC was 4.2% while the incidence in the Cancer Center clinic at UF was 1.7%. In the latter two groups, patients had mostly solid tumors. The results described herein are consistent with the reports of increased incidence of BONJ with greater duration of therapy and that myeloma patients are more likely to develop the complication than solid tumor patients. The results described herein did not identify other specific predictive risk factors for developing BONJ.

Table 2. Demographics of BONJ patients, malignancy, therapy and time to diagnosis of ONJ

| | N = 27 |
|---|---|
| Demographics | |
| Age, years, mean (SD) | 63.7 (9.7) |
| Race/ethnicity | |
| Caucasians | 73% |
| African Americans | 15% |
| Other | 12% |
| Male | 77% |
| Malignancy | |
| Multiple myeloma | 69% |
| Prostate cancer | 12% |
| Head and neck cancer | 8% |
| other cancer | 11% |
| Bisphosphanate used | |
| zoledronic acid | 42% |
| pamidronate disodium | 8% |
| Both | 50% |
| Time to diagnosis of BONJ | 28.2 (18.2) |
| (months), means (SD) | |
| Dental procedure | 46% |

Blood samples from myeloma patients seen in an outpatient clinic and treated with IV BPs were collected. Whether or not they had documented BONJ was noted. Sixty seven myeloma patients who had been treated with IV BPs were genotyped, of whom 8 had BONJ using 5 SNPs; the results are shown in Table 2. Patients with BONJ were compared to those without, and a trend toward a higher odds ratio in BONJ patients was observed with 2 of the SNPs: TNFRSF11A (rs12458117) (SEQ ID NO:1) and MMP2 (rs243865) (SEQ ID NO:2). Carriers of both SNPs had a 50% event rate, while those subjects who did not carry either SNP had an event rate of 7.9%. The odds ratio (95% confidence interval) was 11.6 (1.34-100.8).

TABLE 3
Preliminary results of 5 SNPs and their association with BONJ

| | | | | | Variant carrier vs. |
|---|---|---|---|---|---|
| | | | | | wild-type |
| | | Minor | | HWE | Odds Ratio |
| Gene | SNP | Allele | freq | p | (95% CI) |
| CYP2C8 | rs1934980 | C | 17.40% | 0.15 | 0.93 (0.15-5.72) |
| | (SEQ ID | | | | |
| | NO: 13) | | | | |
| CYP2C8 | rs1934951 | A | 22.30% | 0.77 | 0.71 (0.12-4.30) |
| | (SEQ ID | | | | |
| | NO: 14) | | | | |
| COL1A1 | rs1800012 | G | 7.60% | 0.58 | 1.13 (0.11-11.48) |
| | (SEQ ID | | | | |
| | NO: 15) | | | | |
| TNFRSF11A | rs12458117 | A | 13.00% | 0.78 | 1.72 (0.27-10.98) |
| | (SEQ ID | | | | |
| | NO: 1) | | | | |
| MMP2 | rs243865 | T | 16.40% | 0.47 | 1.75 (0.36-8.63) |
| | (SEQ ID | | | | |
| | NO: 2) | | | | |

Preliminary results on the protein levels by BONJ status: Using Luminex's xMAP technology, serum samples from these 67 patients were analyzed for the protein levels of several factors known to be involved in bone homeostasis; results are shown in Table 4 below. Parathyroid hormone (PTH), Insulin, TNF-alpha, leptin, osteocalcin (OC), osteoprotegerin (OPG), osteopontin (OPN) and IL-6 were studied. A significantly higher serum level of (OPN) was seen in patients with BONJ compared with controls (p=0.037). A higher level of TNF alpha was also noted (p=0.06).

TABLE 4
Protein levels in patients with or without BONJ.

| | PTH | Insulin | TNF alpha | Leptin | OC | OPG | OPN | IL6 |
|---|---|---|---|---|---|---|---|---|
| | (SEQ ID | (SEQ ID | (SEQ ID | (SEQ ID | (SEQ ID | (SEQ ID | (SEQ ID | (SEQ ID |
| | NO: 16) | NO: 17) | NO: 18) | NO: 19) | NO: 20) | NO: 21) | NO: 22) | NO: 23) |
| BONJ | (pg/ml) | (pg/ml) | (pg/ml) | (pg/ml) | (pg/ml) | (pg/ml) | (pg/ml) | (pg/ml) |
| Yes | 96.2 | 375.5 | 6.9 | 2728.3 | 3341.6 | 476.0 | 6104.3 | 38.2 |
| (N = 8) | (48.4) | (314.4) | (5.5) | (2713) | (1315.8) | (267.9) | (7457) | (59.9) |
| NO | 64.4 | 1063.3 | 3.5 | 1898.7 | 2656.5 | 385.1 | 1637.5 | 15.1 |
| (N = 59) | (42.5) | (1267.9) | (2.2) | (1729.8) | (1356.5) | (158.3) | (3464) | (25.5) |
| p value for | 0.16 | 0.35 | 0.06 | 0.46 | 0.28 | 0.47 | 0.037 | 0.27 |
| Wilcoxon | | | | | | | | |
| test | | | | | | | | |

These results have demonstrated that the genes TNFRSF11A and MMP2, as well as the bone protein OPN, are significantly involved in osteoclastogenesis, resorption and skeletal homeostasis. Therefore, it is likely they are implicated in the pathophysiology of BONJ.

### Other Embodiments

Any improvement may be made in part or all of the kits and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

1. A method of identifying a subject having a predisposition to bisphosphonate-induced jaw osteonecrosis (BONJ) following bisphosphonate administration, the method comprising the steps of:
(a) obtaining a sample from the subject;
(b) analyzing the sample for the presence of at least one gene having an SNP that is a biomarker for BONJ or a predisposition to BONJ, a protein encoded by the at least one gene, or at least one SNP that is a marker for BONJ or a predisposition to BONJ; and
(c) correlating the presence of the gene, protein or SNP that is a marker for BONJ or a predisposition to BONJ in the sample with a predisposition to BONJ in the subject.

2. The method of claim 1, wherein the sample comprises blood, serum, plasma or saliva.

3. The method of claim 1, wherein the at least one SNP is rs12458117 or rs243865.

4. The method of claim 1, wherein step (b) of analyzing the sample for the presence of at least one gene having an SNP that is a biomarker for BONJ or a predisposition to BONJ, a protein encoded by the at least one gene, or at least one SNP that is a marker for BONJ or a predisposition to BONJ comprises use of a microarray to detect the presence of the at least one gene or at least one SNP.

5. A method of preventing BONJ in a subject having a predisposition to BONJ and receiving bisphosphonate therapy, the method comprising the steps of:
(a) obtaining a sample from the subject;
(b) analyzing the sample for the presence of at least one gene having an SNP that is a biomarker for BONJ or a predisposition to BONJ; a protein encoded by the gene, or at least one SNP that is a biomarker for BONJ or a predisposition to BONJ;
(c) correlating the presence of the at least one gene, protein, or at least one SNP that is a biomarker for BONJ or a predisposition to BONJ in the sample with a predisposition to BONJ in the subject; and
(d) administering to the subject a bisphosphonate that is not associated with BONJ or that is less likely to cause BONJ than other bisphosphonates, administering to the subject a bisphosphonate at a lower dose or lower frequency than what is conventionally prescribed.

6. The method of claim 5, wherein the sample comprises blood, serum, plasma or saliva.

7. The method of claim 5, wherein the at least one SNP is rs12458117 or rs243865.

8. The method of claim 5, wherein step (b) of analyzing the sample for the presence of at least one gene having an SNP that is a biomarker for BONJ or a predisposition to BONJ, a protein encoded by the at least one gene, or at least one SNP that is a marker for BONJ or a predisposition to BONJ comprises use of a microarray to detect the presence of the at least one gene or at least one SNP.

9. A kit for identifying patients who have a predisposition to BONJ following biphosphonate administration, the kit comprising:
(a) a solid support having a plurality of nucleic acids adhered thereto, wherein at least one of the nucleic acids specifically hybridizes to a gene having an SNP that is a biomarker for BONJ or a predisposition to BONJ;
(b) a detection reagent; and
(c) instructions for use.

10. The kit of claim 9, wherein the at least one gene is a gene set forth in Table 1 or Table 3.

11. The kit of claim 9, wherein the SNP is an SNP set forth in Table 1 or Table 3.

12. A method for assessing a subject's risk of developing BONJ following bisphosphonate treatment comprising: a) obtaining a biological sample from the subject; b) detecting one or more BONJ-associated biomarkers in said sample, wherein the biomarkers are related to one or more genes set forth in Table 1 or Table 3, or said biomarkers are related to one or more polypeptides encoded by said genes resulting in a biomarker data set; c) comparing the biomarker data set to biomarker data from healthy people and people having BONJ; and d) determining the subject's risk of developing BONJ.

13. The method according to claim 12, wherein at least one biomarker is an SNP residing in a gene set forth in Table 1 or Table 3.

14. The method according to claim 12, wherein at least one biomarker is a BONJ-associated polymorphic site associated with one or more of the SNP markers set forth in Table 1 or Table 3.

15. The method according to claim 12, wherein at least one biomarker is an SNP being in complete linkage disequilibrium with one or more of the SNP markers set forth in Table 1 or Table 3.

16. The method according to claim 12, wherein at least one biomarker is an expression product of a gene set forth in Table 1 or Table 3.

17. A method of identifying a subject having a predisposition to BONJ following bisphosphonate administration, the method comprising the steps of:
(a) obtaining a sample from the subject;
(b) analyzing the sample for expression of a protein involved in bone homeostasis; and
(c) correlating expression of the protein with a predisposition to BONJ in the subject.

18. The method of claim 17, wherein the protein involved in bone homeostasis is selected from the group consisting of: PTH, insulin, TNF-α, leptin, OPN, OC, OPG and IL6.

19. The method of claim 17, wherein the sample is analyzed for overexpression of the protein.

20. The method of claim 17, wherein the protein shares at least 90% amino acid sequence identity with a protein listed in Table 4.
